(19) United States
(12) Patent Application Publication
(10) Pub. No.: US 2011/0305654 A1
(43) Pub. Date: Dec. 15, 2011

(54) ANHYDROUS PETROLEUM JELLY/ELASTOMER-FREE DEPIGMENTING COMPOSITIONS COMPRISING A SOLUBILIZED PHENOLIC COMPOUND

(75) Inventors: Claire Mallard, Mougins (FR); Karine Nadau-Fourcade, Villeneuve-Loubet (FR); Fabienne Louis, Villeneuve-Loubet (FR)

(73) Assignee: Galderma Research & Development, Biot (FR)

(21) Appl. No.: 12/994,886

(22) PCT Filed: Jun. 2, 2009
(86) PCT No.: PCT/FR2009/051037

§ 371 Date: Feb. 9, 2011

(30) Foreign Application Priority Data
May 30, 2008 (FR) 0853571

(51) Int. Cl. A61K 8/92; A61Q 19/02; A61K 8/97

(52) U.S. Cl. 424/62

(57) ABSTRACT

Novel dermatological depigmenting compositions, especially for topical application, contain as pharmaceutical active agents a dissolved phenolic compound, formulated as anhydrous compositions free of petroleum jelly and free of elastomer.

The present invention relates to a novel cosmetic or pharmaceutical depigmenting composition in the form of an anhydrous ointment not containing any petroleum jelly and free of elastomer having a high molecular weight, especially for topical application, comprising as a pharmaceutical active agent a dissolved phenolic derivative.

Among the therapeutic agents recommended in the treatment of cutaneous hyperpigmentation, phenolic derivatives, and more particularly polyphenols, have for decades been among the active agents that are the most effective. The therapeutic use of these agents results from the observation of cutaneous depigmentations in the case of operatives in the rubber industry, in which some of these products are used as antioxidants. Subsequently, numerous studies have confirmed their efficacy, alone or combined with other depigmenting agents [Jorge L. Sanchez, M.D. and Miguel Vazquez, M.D. International Journal of Dermatology January-February 1982 Vol. 21, pp. 55-58]. They are thus found to be active agents that are virtually indispensable in the treatment of hyperpigmentation and are consequently present in many commercial products.

Among the phenolic derivatives, polyphenols such as hydroquinone are the pharmaceutical active agents most commonly used. Hydroquinone has been the subject of filing of various patent applications, and in particular U.S. Pat. No. 3,856,934 in which hydroquinone is in combination with retinoic acid and a corticoid as a depigmenting composition.

Rucinol or lucinol, or 4-butylresorcinol, is also a phenolic-based pharmaceutical active agent, of polyphenol type, sold as an agent for lightening brown marks associated with pigmentation disorders (the product Iklen®).

Phenolic derivatives thus appear as virtually unavoidable active agents in the treatment of hyperpigmentation and are consequently present in many commercial products.

However, in the majority of cases, hydroquinone, rucinol or salts or derivatives thereof are dissolved in the aqueous phase of the preparation.

It is known that a certain number of active principles with advantageous therapeutic activity are sensitive to oxidation and especially undergo chemical degradation leading to a substantial loss of their activity in the presence of water. The incorporation of a phenolic derivative such as hydroquinone or rucinol is thus a major drawback in this type of aqueous preparation.

Specifically, degradation of formulations containing phenolic derivatives such as hydroquinone or rucinol, alone or in combination with other active principles, is often observed. These active agents are effectively known for their great sensitivity to oxidation and to heat, leading to a reduction in efficacy, rapid browning of the formulations and occasionally even demixing of the formulation.

Furthermore, to accelerate their solubilization, phenolic derivatives such as hydroquinone or rucinol are often exposed to heat during the phase of preparing the composition, especially in standard emulsions, and this phenomenon initiates and accelerates the browning.

In the prior art, reducing agents are used to combat this degradation, in particular sulfites, which are virtually indispensable. However, these antioxidants have a certain number of drawbacks (odour, irritation, allergenic power).

The second drawback due to the presence of phenolic derivatives such as hydroquinone, alone or in combination with other active agents in the composition, is their strong irritant power.

As a result of its irritant power, hydroquinone at high concentration can give rise to post-inflammatory hypermelanosis and ochronosis phenomena.

Local irritation and dermatitis may develop after a prolonged use of hydroquinone at high concentration [“N-acetyl-4S cysteaminylphenol as a new type of depigmenting agent” Jimbow K. Arch. Dermatol. 1991 October; 127 (10): 1528-1534].

Treatment with hydroquinone may be accompanied by irritation that may lead to a post-inflammatory hyperpigmentation. The incidence of the irritation depends on the hydroquinone concentration. This irritation is relatively high for 10% concentrations and reduces greatly for preparations with a 5% dose, and is considered to be virtually nonexistent at a concentration of 2% [“Les agents chimiques dépigmentants (Depigmenting chemical agents)” JP. Ortonne Ann. Dermatol. Venerol. 1986, 113: 733-736].

The chosen galenical form may thus play a predominant role in minimizing these effects.

Consequently, in order to avoid the presence of sulfites and/or eliminate or limit the use of antioxidants, phenolic-based pharmaceutical active agents and in particular hydroquinone or rucinol should be formulated in solubilized form in an anhydrous formulation.

For comfort of use, it is important for an ointment free of petroleum jelly and free of elastomer to have, however, a sufficiently high viscosity.

The anhydrous compositions currently available on the market, or as described in patent application US 2006/0 120 979 and allowing the formulation of water-sensitive active principles, while at the same time affording them good chemical stability, are generally compositions of ointment type, formed mainly from petroleum jelly or, in more recent formulations, from a large proportion of elastomer.

The use of petroleum jelly is unsatisfactory for the following reasons:
after application, certain compositions comprising petroleum jelly are experienced as being tacky and greasy, and furthermore are glossy;
moreover, the preparation of compositions in the form of petroleum jelly-based ointments requires particular compounds and conditions. The reason for this is that petroleum jelly is solid at room temperature, and has a melting point above 40° C. In order to be able to mix it with other compounds, it is necessary to formulate it in liquid form, and thus to manufacture the compositions at temperatures above 40° C. However, such a process has the drawback of forming a crust. Specifically, faster cooling of the exterior of the composition relative to its core causes its abnormal hardening (crusting), which has the effect of slowing down or even of preventing perfect homogenization from being obtained;
finally, the formulation of phenolic derivatives, especially hydroquinone or rucinol, is difficult on account of the sensitivity of these active agents to heat.

The use of elastomer in relatively large amounts makes it possible to give a certain amount of viscosity to anhydrous formulations (patent US 2006/0 120 979) without the drawbacks of petroleum jelly. However, in the present invention, its use is unsatisfactory for the following reasons: the high proportion of elastomer present in these formulations specifically prevents the incorporation of sufficient amounts of oily and waxy compounds that have the advantage of giving the preparation the desired emollient properties.

One of the aims of the present invention is to propose an anhydrous pharmaceutical composition free of petroleum jelly and free of elastomer, intended for topical application, which has a viscosity equivalent to that of ointments containing petroleum jelly, which is easy to prepare, which affords good chemical stability to the active agent and in which certain volatile compounds may be used. The composition according to the invention especially has these advantages by virtue of its preparation process. A subject of the invention is thus also the particularly advantageously process for preparing such a composition, in which the step of incorporating the active agent is performed at room temperature. The desired viscosity of the composition according to the invention is obtained especially by means of the choice of fatty substances used. The absence of elastomer makes it possible to obtain the desired feature of the formulation, namely a certain level of fluidity of the composition at the end of manufacture allowing easy incorporation at room temperature and perfect homogenization of the active agents, and then a final viscosity reached about 24 hours after manufacture.

Another aim of the present invention is to propose an anhydrous pharmaceutical composition free of petroleum jelly and free of elastomer, intended for topical application, with prolonged stability, allowing optimized release of the active agents while at the same time being very well tolerated.

The present invention thus relates to a novel stable composition in the form of an anhydrous composition not containing any petroleum jelly and free of elastomer, especially for topical application, comprising a dissolved phenolic-based pharmaceutical active agent of polyphenol type. The anhydrous composition according to the present invention also shows excellent stability and harmlessness.

According to the invention, the term “elastomer” means a polyorganosiloxane elastomer.

The term “anhydrous composition” means a composition comprising an amount of water of less than or equal to 5% by weight relative to the total weight of the composition.

In one preferred mode according to the invention, the composition does not contain any water.

The term “stable composition” means a chemically and physically stable composition.

The term “chemical stability” especially means that no degradation of the active agent is observed over time and at temperatures of between 4 and 40° C. The term “physical stability” especially means that the compositions do not show any drop in viscosity over time and at temperatures between 4 and 40° C.

The subject of the present invention is thus an anhydrous pharmaceutical composition, characterized in that it comprises:

a. at least one pharmaceutical active agent of phenolic derivative type,

b. glyceryl behenate and derivatives or mixtures thereof,

c. at least one solvent for the phenolic derivative,

the said composition not containing any petroleum jelly or any polyorganosiloxane elastomer.

The anhydrous composition according to the invention may be in the various known galenical forms, which a person skilled in the art will adapt to the particular use of the composition.

The compositions according to the invention are preferably formulated for topical application.

The term “topical application” means external application to the skin or mucous membranes.

The compositions according to the invention may be in any galenical form normally used for topical application. As a non-limiting example of compositions as described in the American pharmacopoeias (USP32-NF27—Chap 1151—Pharmaceutical Dosage Forms) or European pharmacopoeias (Edition 6.3—in the chapter: Préparations semi-solides pour application cutanée [Semi-solid preparations for cutaneous application] or as defined in the decision trees of the American Food and Drug Administration (FDA) (CDER Data Standards Manual Definitions for topical dosage Forms). The compositions according to the invention may thus be in liquid, semi-solid, pasty or solid form, and more particularly in the form of ointments, oily solutions, dispersions of the lotion type, which may be two-phase lotions, serum, anhydrous or lipophilic gels, powders, impregnated pads, syndets, wipes, sprays, mousses, sticks, shampoos, compresses, washing bases, emulsions of liquid or semi-liquid consistency of the oil-in-glycol or glycol-in-oil type, a microemulsion, semi-liquid or solid suspensions of the white or coloured cream type, multiple or inverse emulsions, gel or pomade, suspensions of microspheres or nanospheres or of lipid or polymeric vesicles, or microcapsules, microparticles or nanoparticles, or polymeric or gelled patches for controlled release.

The anhydrous composition according to the invention is preferably an ointment. According to the invention, the term “ointment” means a composition especially as defined in the American or European Pharmacopoeias mentioned above. The FDA thus defines an ointment as a semi-solid composition comprising, as vehicle, less than 20% water and volatile compounds and more than 50% hydrocarbons, waxes or polyol. In certain cases, when the contents of volatiles are high, such compositions may be referred to as creams (decision tree of the American Food and Drug Administration (FDA)). The American Pharmacopoeia defines an ointment as being a product whose base is a vehicle that may belong to the following four classes: hydrocarbon base or absorbent base or water-washable base or water-soluble base. In the present invention, the ointment as defined in the American Pharmacopoeia is of hydrocarbon base type. The European Pharmacopoeia defines an ointment as being a one-phase composition in which liquids or solids may be dispersed.

The ointment according to the invention is preferentially a composition that is thick at room temperature, which comprises between 80% and 98% by weight, relative to the total weight of the composition, of hydrophobic compounds other than petroleum jelly. Such compounds are chosen especially from liquid oils alone or as a mixture, the said oils possibly being hydrocarbons, esters, plant oils and/or silicone oils, which are volatile or non-volatile, which may be gelled with lipophilic compounds that are solid at room temperature such as waxes, butters or fatty acid esters.

Optionally, a measurement of the flow threshold may be performed in order to characterize the finished product.

For the measurement of the flow threshold, a VT550 Haake rheometer with an SVDIN measuring spindle was used.

The rheograms are produced at 25° C. and an imposed speed of 0 to 100 s⁻¹. The viscosity values are given at shear values of 4 s⁻¹, 20 s⁻¹, 100 s⁻¹ (γ). The term “flow threshold” (τ₀ expressed in Pascals) means the force (minimum shear stress) required to overcome the cohesion forces of Van der Waals type and to bring about flow.

Throughout the present patent application, the term “room temperature” means a temperature of between 20 and 30° C.

The anhydrous nature of the ointment not containing any petroleum jelly or any elastomer according to the invention makes it possible to avoid instability of the phenolic derivative, in particular its oxidation in aqueous medium. In such a formulation, the use of antioxidants of sulfite type that are essential for the stabilization of hydroquinone in aqueous medium is thus no longer necessary. Consequently, in one preferred mode according to the invention, the composition does not contain any sulfites and contains an amount of antioxidants strictly less than 0.3% and preferentially less than 0.2% by weight relative to the total weight of the composition. The antioxidants that may be used according to the invention are preferably antioxidants such as vitamin E and derivatives thereof, for instance DL-α-tocopherol or tocopheryl acetate from Roche; vitamin C and derivatives thereof, for instance ascorbyl palmitate from Roche, and butylhydroxytoluene sold under the name Nipanox BHT by Clariant.

Thus, the anhydrous ointment according to the invention comprises:
at least one pharmaceutical active agent of dissolved phenolic derivative type,
glyceryl behenate and/or derivatives and/or mixtures thereof,
optionally, at least one additional lipophilic thickener or gelling agent,
at least one solvent for the phenolic derivative, and
optionally at least one fatty substance or oil.

Preferably, as mentioned above, the anhydrous composition according to the invention contains substantially no petroleum jelly, i.e. comprises not more than 1% by weight of petroleum jelly relative to the total weight of the composition.

The term “phenolic-based pharmaceutical active agent” means, in a non-limiting manner, polyphenols and more particularly hydroquinone, 4-hydroxyanisole, hydroquinone monoethyl ether, hydroquinone monobenzyl ether and rucinol or lucinol and salts thereof.

The term “rucinol salts” especially means salts formed with a pharmaceutically acceptable base, especially a mineral base such as sodium hydroxide, potassium hydroxide and aqueous ammonia or an organic base such as lysine, arginine or N-methylglucamine, but also the salts formed with fatty amines such as dioctylamine, aminomethylpropanol and stearylamine.

Hydroquinone or rucinol is preferably used.

Advantageously, the amount of pharmaceutical active agents of phenolic derivative type is from 0.01% to 10% by weight, preferably from 0.05% to 6% by weight and more particularly from 0.01% to 5% by weight relative to the total weight of the composition.

The composition according to the invention comprises glyceryl behenate, derivatives thereof or mixtures thereof. The term “glyceryl behenate derivatives” especially but not exclusively means glyceryl monobehenate, glyceryl dibehenate and tribehenine. The composition according to the invention especially comprises, preferably, a mixture of glyceryl dibehenate, tribehenine and glyceryl behenate. Such a mixture is especially sold under the name Compritol 888 by Gattefosse. In the rest of the description of the invention, the term “glyceryl behenate” will be understood as meaning glyceryl behenate, derivatives thereof or mixtures thereof. Glyceryl behenate is an oily-phase thickener. In the composition according to the invention, the glyceryl behenate sets to a solid over time and allows the preparation of a hydrophobic composition whose final viscosity is obtained only after a certain time. In the particular case according to the invention, the constituents and the process are effectively chosen so as to give the composition fluidity at the end of immediate manufacture, facilitating the homogenization of the various constituents, but a desired final viscosity about 24 hours after manufacture. To obtain this result, the composition comprises from 1% to 40%, preferably between 5% and 30% and even more preferentially from 10% to 25% by weight of glyceryl behenate relative to the total weight of the composition.

The composition according to the invention may also comprise at least one additional lipophilic gelling agent, also known as a lipophilic thickener. Such an additional lipophilic gelling agent or thickener affords greater physical stability to the composition, in particular when the composition is subjected to temperatures of accelerated stability conditions (ICH criteria) at about 40° C. Specifically, these compounds are used in the present invention as “viscosity modifiers”: in particular, by appropriately selecting them, they ensure the stability of the composition at 40° C. This thus affords the compositions obtained better stability.

According to the invention, the term “additional lipophilic thickeners or gelling agents” means compounds different from glyceryl behenate, chosen especially from waxes, hydrogenated oils and fatty acid esters.

The term “wax” generally means a lipophilic compound, which is solid at room temperature (25° C.), with a reversible solid/liquid change of state, which has a melting point of greater than or equal to 30° C., which may be up to 200° C. and especially up to 120° C. As waxes that may be used, mention may be made of carnauba wax, microcrystalline waxes, beeswax, sold under the name Cerabeil blanche by Barlocher, or candelilla wax.

The term “hydrogenated oil” means oils obtained by catalytic hydrogenation of animal or plant oils containing linear or branched C₈-C₃₂ fatty chains. Among these oils, mention may be made especially of hydrogenated jojoba oil, isomerized jojoba oil such as partially hydrogenated trans-isomerized jojoba oil manufactured or sold by the company Desert Whale under the commercial reference Iso-Jojoba-50®, hydrogenated sunflower oil, the hydrogenated castor oil sold especially under the name Cutina HR by Cognis, hydrogenated coconut oil and hydrogenated lanolin oil; hydrogenated castor oil will preferably be used.

As fatty acid esters that may be used, mention may be made of lanolin sold especially under the name Medilan by Croda, the fatty acid esters of glycerol sold under the name Gelucire by Gattefosse, the hydrogenated coconut glycerides sold under the name Akosoft 36 by Karlshamns, or the diethylene glycol or propylene glycol monostearate sold, respectively, under the names Hydrine or Monosteol by Gattefosse.

Thus, preferably, the composition comprises an overall amount of glyceryl behenate and optionally of additional lipophilic thickeners or gelling agents of between 1% and 40% and preferably between 5% and 35% by weight relative to the total weight of the composition. Preferably, the composition comprises from 10% to 25% by weight of glyceryl behenate and from 0 to 30% and preferably from 1% to 10% by weight of additional lipophilic thickener.

According to the invention, the term “composition free of elastomer” means an anhydrous composition comprising not more than 1% by weight of elastomer relative to the total weight of the composition. Preferably, as mentioned above, the ointment according to the invention does not contain any elastomer.

The term “elastomer” means any polyorganosiloxane elastomer, namely any chemically crosslinked siloxane polymer that has viscoelastic properties. Specifically, the desired viscosity of the composition according to the invention is obtained with the aid especially of glyceryl behenate and of the choice of the other fatty substances used. The absence of elastomer in the composition especially makes it possible to introduce more oily compounds, thus giving the composition the desired emollient properties. The absence of elastomer may especially make it possible to obtain a more pronounced effect of the glyceryl behenate, namely fluidity of the composition at the end of manufacture and a final viscosity reached about 24 hours after manufacture.

The composition also comprises at least one solvent for the phenolic-based pharmaceutical active agent. Solvents for the phenolic derivative are especially solvents of alcoholic or glycolic type.

Examples of solvents of alcoholic type according to the invention that may be mentioned include linear or branched aliphatic alcohols, such as anhydrous or non-anhydrous ethanol, isopropanol and butanol. The composition according to the invention preferentially contains ethanol.

Examples of solvents of glycolic type according to the invention that may be mentioned include propylene glycol, ethylene glycol, 1,3-butylene glycol and dipropylene glycol. The solvents for the phenolic derivative of alcoholic or glycolic type that are preferred according to the invention are especially ethanol and propylene glycol.

According to one of the preferred modes according to the invention, the solvent for the phenolic-based pharmaceutical active agent is ethanol.

Preferably, the total amount of solvent is between 1% and 80% by weight, preferably between 5% and 50% and more particularly, between 10% and 30% by weight relative to the total weight of the composition.

In addition to the alcoholic or glycolic solvent, and in order to prepare a composition having the desired properties, for example in terms of consistency, texture or emollient or moisturizing qualities, a person skilled in the art may add one or more fatty substances chosen from the following list:
plant oils, such as the sweet almond oil sold by Sictia or the sesame oil sold by CPF,
silicone oils such as the cyclomethicone sold under the name ST-Cyclomethicone 5NF by Dow Corning or the dimethicone sold under the name Q7 9120 Silicone

### Fluid by Dow Corning,

mineral oils, such as Marcol 152 or Primol 352 sold by Esso,
perhydrosqualene,
triglycerides, such as the caprylic/capric triglycerides sold under the name Miglyol 812 N by IMCD, or derivatives such as PEG-8 caprylic/capric triglycerides sold under the name Labrasol by Gattefossé,
esters, such as the octyldodecyl myristate sold under the name MOD by Gattefossé, the C₁₂-C₁₅ alkyl benzoate sold under the name Tegosoft TN by Goldschmidt or the cetearyl isononanoate sold under the name Cetiol SN PH by Cognis,
Guerbet alcohols, such as the octyldodecanol sold under the name Eutanol G by Cognis,
ethers and derivatives, such as the PPG-15 stearyl ether sold under the name Arlamol E by Croda,
and mixtures thereof.

When at least one oil is present in the composition, the amount thereof is between 0.05% and 98% by weight and preferably between 1% and 80% by weight.

Optionally, the composition according to the invention may also comprise at least one surfactant and/or at least one binder.

The surfactants used are preferably nonionic surfactants, which are used for example, but not exclusively, to facilitate the incorporation of certain constituents such as glycols into the oily phase of the composition.

Among the surfactants that may be used according to the invention, mention may be made of esters of glycerol and optionally of polyethylene glycol, such as the mixture of glyceryl stearate and of PEG-100 stearate, sold under the name Arlacel 165 by Uniqema, the mixture of glyceryl stearate and of PEG-75 stearate sold under the name Gelot 64 by Gattefossé, the glyceryl stearate sold under the name Cutina GMSV by Cognis; emulsifying waxes, such as the self-emulsifying wax sold under the name Polawax NF by Croda or the PEG-8 beeswax sold under the name Apifil by Gattefossé; the polysorbate 80 sold under the name Tween 80 by Uniqema; castor oil and derivatives, for instance the polyoxyethylenated castor oil from BASF sold under the trade name Cremophor EL or the mixture of glyceryl stearate and PEG-2 stearate, sold under the name Sedefos 75 by Gattefossé.

The amount of surfactants is between 1% and 20% by weight and preferably between 1% and 10% by weight. The composition may optionally comprise at least one binder. Among the binders that may be used, mention may be made of the magnesium stearate sold by Brenntag, the corn starch sold by Roquette, the talc sold by WCD, the cholesterol sold by Croda or the silica sold by Degussa.

The binders may be used in an amount of between 1% and 30% by weight and preferably between 1% and 20% by weight.

The composition according to the invention may also contain additives at between 0 and 20% and preferably between 0 and 10% by weight relative to the total weight of the composition, these being additives that a person skilled in the art will select as a function of the desired effect.

Among the additives, examples that may be mentioned include, taken alone or in combination:
vitamins such as vitamin PP or niacinamide,
calmatives or anti-irritant agents such as PPG-12/SMDI copolymer sold by the company Bertek Pharmaceuticals under the trade name Polyolprepolymer-2 or glycyrrhetinic acid or derivatives thereof, for instance Enoxolone sold by Cognis, or hyaluronic acid,
moisturizers or humectants: examples that may be mentioned include sugars and derivatives, glycols, glycerol and sorbitol,
lecithins and cholesterol,
preserving agents, such as the methyl paraben sold under the name Nipagin M by Clariant, the propyl paraben sold under the name Nipasol by Clariant, or the phenoxyethanol sold under the name Phenoxetol by Clariant,
acids or bases such as citric acid, sodium citrate, triethanolamine, aminomethylpropanol, sodium hydroxide and diisopropanolamine,
other additives for giving the said preparation specific properties.

Preferentially, the composition according to the invention comprises, on a weight basis relative to the total weight:
0.01% to 10% of at least one pharmaceutical active agent of phenolic derivative type,
1% to 40% of glyceryl behenate,
1% to 80% of at least one ethanolic or glycolic solvent,
0 to 30% of additional lipophilic thickeners,
0.05% to 98% of oils,
0 to 20% of additives.

More preferentially, the composition according to the invention comprises, on a weight basis relative to the total weight:
0.01% to 10% of at least one phenolic-based pharmaceutical active agent, preferably hydroquinone or rucinol,
5% to 30% of glyceryl behenate,
5% to 50% of at least one ethanolic or glycolic solvent,
1% to 10% of additional lipophilic thickeners,
1% to 80% of oils,
0 to 20% of surfactants,
0 to 30% of binder(s),
0 to 10% of additives.

Even more preferentially, the composition according to the invention comprises, on a weight basis relative to the total weight:
0.01% to 6% of hydroquinone or rucinol,
10% to 25% of glyceryl behenate,
1% to 10% of additional lipophilic thickeners,
10% to 30% of ethanol,
1% to 80% of oils,
0 to 10% of surfactants,
0 to 20% of binder(s),
0 to 10% of additives.

A subject of the invention is also the use of the composition thus obtained, as a medicament.

More particularly, the composition may be used for preparing a medicament intended for treating and preventing hyperpigmentary disorders such as melasma, chloasma, lentigo, senile lentigo, vitiligo, freckles, post-inflammatory hyperpigmentations caused by abrasion, a burn, a scar, a dermatitis or a contact allergy; naevi, genetically determined hyper-pigmentations, hyperpigmentations of metabolic or medicinal origin, melanomas or any other hyperpigmentary lesions.

A subject of the invention is also the use of the composition in the cosmetic field.

The compositions according to the invention also find an application in the cosmetic field, in particular in protecting against the harmful effects of sunlight, for preventing and/or combating light-induced or chronological ageing of the skin and the integuments.

The invention also relates to a non-therapeutic cosmetic treatment process for beautifying the skin and/or for improving its surface appearance, characterized in that a composition comprising at least one depigmenting agent is applied to the skin and/or its integuments.

Finally, a subject of the present invention is also a process for preparing the compositions according to the invention. Such a process especially makes it possible to maintain the compounds in fluid form at the end of manufacture. One of the essential characteristics of the process for preparing the compositions according to the invention is the incorporation of the active phase at room temperature, i.e. the final step of mixing of the phases is performed at room temperature.

The term “room temperature” means a temperature of between 20 and 30° C.

In the process according to the invention, the term “active phase” means a phase containing at least one active principle. Similarly, in the process according to the invention, the term “non-active phase” means a phase formed from any ingredient other than the active principle. In the composition according to the invention, the non-active phase is preferentially an oily phase containing at least the glyceryl behenate, preferably with another oily compound as described previously.

Advantageously, the process for manufacturing the composition according to the invention is performed according to Example 1, characterized in that the phases containing the pharmaceutical active agents are mixed together at room temperature.

The process gives the product the following advantages:
good homogeneity of the active agents since all the components are mixed in a fluid phase,
the absence of crusting during cooling and good fluidity of the product up to the end of manufacture,
easy packaging due to the low viscosity at the end of manufacture, the final viscosity of the composition of ointment type not being reached immediately at the end of manufacture,
the mixing carried out at room temperature avoids the volatilization of the solvent(s) and the degradation of the heat-sensitive active agent, and especially the phenolic derivative such as hydroquinone or rucinol.

The formulation examples below illustrate the compositions according to the invention without, however, limiting its scope. The amounts of the constituents are expressed as weight percentages relative to the total weight of the composition.

For all the formulations, the physical stability is measured by macroscopic and microscopic observation of the formulation at room temperature, at 4° C. and at 40° C. after 1 month, 2 months and optionally 3 months.

The macroscopic observation makes it possible to ensure the physical integrity of the products and the microscopic observation makes it possible to check that there is no recrystallization of the dissolved active agent.

The chemical stability is measured by assaying the active agents by external calibration on HPLC, and the results are expressed as a percentage of recovery relative to the theoretical titre.

### EXAMPLE 1

### Process for Preparing the Compositions

a) Preparation of the Fatty Phase or Non-Active Phase (Phase A):

Introduce all the constituents, consistency factors and oils into the manufacturing beaker. Stir at elevated temperature to obtain uniform melting of the ingredients. Stop the heating.

Add the additives of the fatty phase, if necessary, then cool to room temperature with stirring.

b) Active Phase (phase B):

Dissolve the phenolic-based pharmaceutical active agent in the appropriate solvent, add one (or more) antioxidant(s), if necessary, and stir until the active agent has dissolved (phase B).

c) Preparation of the Final Composition:

Incorporate, with stirring, the active phase into the formulation base at room temperature, for the solubilization in ethanolic or glycolic phase.

Incorporate the additional phases if necessary.

Homogenize and continue cooling with stirring.

Packaging is performed at the end of manufacture since the product does not yet have its final viscosity.

### EXAMPLE 2

| Phases | INCI name | Formulation % |
|---|---|---|
| A | Glyceryl behenate | 16.00 |
| A | Hydrogenated castor oil | 2.00 |
| A | Caprylic/capric triglycerides | 49.85 |
| A | PEG-8 caprylic/capric | 3.00 |
| | glycerides | |
| A | PPG-15 stearyl ether | 5.00 |
| B | Ethanol 100 | 20.00 |
| B | DL-α-tocopherol | 0.05 |
| B | Ascorbyl palmitate | 0.1 |
| B | Hydroquinone | 4.00 |

Specifications at T0

Macroscopic appearance: Glossy white ointment

Microscopic appearance: Absence of hydroquinone crystals

Haake profile (4 s⁻¹/20 s⁻¹/100 s⁻¹): 31/71/164

Physical Stability:

The composition of Example 2 shows good colour stability (absence of oxidation) for at least 3 months at room temperature, 40° C. and 4° C.

Chemical Stability

[symbol] Hydroquinone

| | |
|---|---|
| Stability | Time |

| conditions | T0 | T + 1 M | T + 2 M | T + 3 M |
|---|---|---|---|---|
| RT | 103.2 | 100.1 | 95 | 99 |
| 4° C. | NA | 97.5 | 96.5 | 100.5 |
| 40° C. | NA | 101.4 | 98 | 100.5 |

### EXAMPLE 3

| Phases | INCI name | Formulation % |
|---|---|---|
| A | Glyceryl behenate | 16.00 |
| A | Hydrogenated castor oil | 2.00 |
| A | Caprylic/capric triglyceride | 57.85 |
| B | Ethanol 100 | 20.00 |
| B | DL-α-tocopherol | 0.05 |
| B | Ascorbyl palmitate | 0.10 |
| B | Hydroquinone | 4.00 |

Specifications at T0

Macroscopic appearance: Glossy white ointment

Microscopic appearance: Absence of hydroquinone crystals.

Haake profile (4 s^{-1/20} s^{-1/100} s⁻¹): 297/501/280

Physical Stability:

| | | T + 1 month | T + 2 months | T + 3 months |
|---|---|---|---|---|
| Macroscopic | RT | In accordance | In accordance | In accordance |
| appearance | | with the | with the | with the |
| | | specifications | specifications | specifications |
| | 40° C. | In accordance | In accordance | In accordance |
| | | with the | with the | with the |
| | | specifications | specifications | specifications |
| | 4° C. | In accordance | In accordance | In accordance |
| | | with the | with the | with the |
| | | specifications | specifications | specifications |
| Microscopic | RT | In accordance | In accordance | In accordance |
| appearance | | with the | with the | with the |
| | | specifications | specifications | specifications |
| | 40° C. | In accordance | In accordance | In accordance |
| | | with the | with the | with the |
| | | specifications | specifications | specifications |
| | 4° C. | In accordance | In accordance | In accordance |
| | | with the | with the | with the |
| | | specifications | specifications | specifications |

| | | | |
|---|---|---|---|
| Haake rheology | 69/254/251 | 294/522/367 | 363/513/395 |
| (4 s⁻¹/20 s⁻¹/100 s⁻¹) | | | |

Chemical Stability:

[symbol] Hydroquinone

| | |
|---|---|
| Stability | Time |

| conditions | T0 | T + 1 M | T + 2 M | T + 3 M |
|---|---|---|---|---|
| RT | 103.7 | 102.9 | 96.75 | 101.3 |
| 4° C. | NA | 103.8 | 99 | 105 |
| 40° C. | NA | 103.2 | 97.5 | 100 |

### EXAMPLE 4

| Phases | INCI name | Formulation % |
|---|---|---|
| A | Glyceryl behenate | 16.00 |
| A | Hydrogenated castor oil | 2.00 |
| A | Caprylic/capric triglycerides | 42.85 |
| B | PPG-15 stearyl ether | 15.00 |
| B | Ethanol 100 | 20.00 |
| B | DL-α-tocopherol | 0.05 |
| B | Ascorbyl palmitate | 0.10 |
| B | Hydroquinone | 4.00 |

Specifications at T0

Macroscopic appearance: Shiny white ointment

Microscopic appearance: Absence of hydroquinone crystals

Haake profile (4 s⁻¹/20 s⁻¹/100 s⁻¹): 255/328/253

Physical Stability:

| | | T + 1 month | T + 2 months | T + 3 months |
|---|---|---|---|---|
| Macroscopic | RT | In accordance | In accordance | In accordance |
| aspect | | with the | with the | with the |
| | | specifications | specifications | specifications |
| | 40° C. | In accordance | In accordance | In accordance |
| | | with the | with the | with the |
| | | specifications | specifications | specifications |
| | 4° C. | In accordance | In accordance | In accordance |
| | | with the | with the | with the |
| | | specifications | specifications | specifications |
| Microscopic | RT | In accordance | In accordance | In accordance |
| aspect | | with the | with the | with the |
| | | specifications | specifications | specifications |
| | 40° C. | In accordance | In accordance | In accordance |
| | | with the | with the | with the |
| | | specifications | specifications | specifications |
| | 4° C. | In accordance | In accordance | In accordance |
| | | with the | with the | with the |
| | | specifications | specifications | specifications |

| | | | |
|---|---|---|---|
| Haake rheology | 378/589/383 | 274/508/341 | 297/579/375 |
| (4 s⁻¹/20 s⁻¹/100 s⁻¹) | | | |

Chemical Stability:

[symbol] Hydroquinone

| | |
|---|---|
| Stability | Time |

| conditions | T0 | T + 1 M | T + 2 M | T + 3 M |
|---|---|---|---|---|
| RT | 105.6 | 106.9 | 99.5 | 100.5 |
| 4° C. | NA | 105.1 | 100 | 98.3 |
| 40° C. | NA | 103.2 | 98 | 104.5 |

### EXAMPLE 5

| Phases | INCI name | Formulation % |
|---|---|---|
| A | Glyceryl behenate | 16.00 |
| A | Hydrogenated castor oil | 2.00 |
| A | Caprylic/capric triglycerides | 48.85 |
| A | PPG-15 stearyl ether | 5.00 |
| B | Ethanol 100 | 20.00 |
| B | DL-α-tocopherol | 0.05 |
| B | Ascorbyl palmitate | 0.10 |
| B | Rucinol | 5.00 |

Specifications at T0

Macroscopic appearance: Glossy white ointment

Microscopic appearance: Absence of rucinol crystals

Haake profile (4⁻¹/20 s⁻¹/100 s⁻¹): 55/57/99

Physical Stability:

| | | T + 1 month | T + 2 months |
|---|---|---|---|
| Macroscopic | RT | In accordance | In accordance |
| appearance | | with the | with the |
| | | specifications | specifications |
| | 40° C. | In accordance | In accordance |
| | | with the | with the |
| | | specifications | specifications |
| | 4° C. | In accordance | In accordance |
| | | with the | with the |
| | | specifications | specifications |
| Microscopic | RT | In accordance | In accordance |
| appearance | | with the | with the |
| | | specifications | specifications |
| | 40° C. | In accordance | In accordance |
| | | with the | with the |
| | | specifications | specifications |
| | 4° C. | In accordance | In accordance |
| | | with the | with the |
| | | specifications | specifications |

| | | |
|---|---|---|
| Haake rheology | 191/307/270 | 202/403/296 |
| (4 s⁻¹/20 s⁻¹/100 s⁻¹) | | |

Chemical Stability:

[symbol] Rucinol

| Stability | Time | | |
|---|---|---|---|
| conditions | T0 | T + 1 M | T + 2 M |
| RT | 102.3 | 101.8 | 102.2 |
| 4° C. | NA | 103.8 | 100.0 |
| 40° C. | NA | 102.4 | 105.6 |

1.-12. (canceled)

13. An anhydrous pharmaceutical composition, comprising:
a. at least one phenolic compound selected from among hydroquinone, rucinol or lucinol, 4-hydroxyanisole, hydroquinone monoethyl ether and hydroquinone monobenzyl ether,
b. glyceryl behenate, or compounds or mixtures thereof,
c. at least one alcoholic or glycolic solvent for the phenolic compound, with the proviso that said composition is devoid of petroleum jelly and devoid of polyorganosiloxane elastomer.

14. The anhydrous pharmaceutical composition as defined by claim 13, wherein the phenolic compound is present in an amount ranging from 0.00001% to 10% by weight relative to the total weight of the composition.

15. The anhydrous pharmaceutical composition as defined by claim 13, wherein the phenolic compound comprises hydroquinone or rucinol.

16. The anhydrous pharmaceutical composition as defined by claim 13, comprising glyceryl behenate in an amount ranging from 1% to 40% by weight relative to the total weight of the composition.

17. The anhydrous pharmaceutical composition as defined by claim 13, further comprising at least one lipophilic thickener and/or at least one surfactant and/or at least one oil and/or at least one binder.

18. The anhydrous pharmaceutical composition as defined by claim 13, comprising an additional lipophilic thickener selected from among oleyl alcohol, cetyl alcohol, cetearyl alcohol, stearyl alcohol, hydrogenated jojoba oil, hydrogenated sunflower oil, hydrogenated castor oil, hydrogenated coconut oil, hydrogenated lanolin oil, lanolin, fatty acid esters of glycerol, hydrogenated coconut glycerides and diethylene glycol or propylene glycol monostearate.

19. The anhydrous pharmaceutical composition as defined by claim 18, comprising an oil selected from among plant oils, mineral oils, silicone oils, caprylic/capric triglycerides, octyldodecyl myristate, C12-C15 alkyl benzoates and cetearyl isononanoate, and mixtures thereof.

20. The anhydrous pharmaceutical composition as defined by claim 13, comprising, on a weight basis relative to the total weight of the composition:
a. 0.01% to 10% of at least one phenolic compound,
b. 1% to 40% of glyceryl behenate,
c. 1% to 80% of at least one ethanolic or glycolic solvent,
d. 0.05% to 98% of fatty substance or oil,
e. 0 to 10% of additional lipophilic thickener or gelling agent,
f. 0 to 20% of additives.

21. The anhydrous pharmaceutical composition as defined by claim 13, comprising, on a weight basis relative to the total weight of the composition:
a. 0.0001% to 6% of hydroquinone or rucinol,
b. 10% to 25% of glyceryl behenate,
c. 10% to 30% of ethanol,
d. 1% to 80% of oil,
e. 0 to 20% of surfactants,
f. 0% to 10% of binder,
g. 0 to 10% of additives.

22. A medicament comprising an anhydrous pharmaceutical as defined by claim 13.

23. A medicament useful for treating and/or preventing hyperpigmentary disorders, melasma, chloasma, lentigo, senile lentigo, vitiligo, freckles, post-inflammatory hyperpigmentations due to abrasion, a burn, a scar, a dermatitis or a contact allergy; naevi, genetically determined hyperpigmentations, hyperpigmentations of metabolic or medicinal origin, melanomas or any other hyperpigmentary lesions, comprising an anhydrous pharmaceutical composition as defined by claim 13.

24. A process for formulating an anhydrous pharmaceutical composition as defined by claim 13, comprising at least the following steps:
a. preparing one or more non-active phases by mixing at least glyceryl behenate with the other constituents of the phase,
b. preparing the active phase by mixing at least one phenolic compound with a solvent therefor,
c. mixing of the active phases with the non-active phase to obtain a homogeneous composition, and wherein the final step c. of mixing of the phases is carried out at room temperature and the said phases are fluid in the final step c.
